(19) United States
(12) Patent Application Publication
(10) Pub. No.: US 2013/0338026 A1
(43) Pub. Date: Dec. 19, 2013

(54) Prognostic Marker for Endometrial Carcinoma

(71) Applicants: Scott L. Carter, Cambridge, MA (US); Rameen Beroukhim, Brookline, MA (US); Helga B. Salvesen, Paradis (NO); Lars A. Akslen, Soreidgrend (NO); Jone Trovik, Bergen (NO); Henrica Maria Johanna Werner, Bergen (NO)

(72) Inventors: Scott L. Carter, Cambridge, MA (US); Rameen Beroukhim, Brookline, MA (US); Helga B. Salvesen, Paradis (NO); Lars A. Akslen, Soreidgrend (NO); Jone Trovik, Bergen (NO); Henrica Maria Johanna Werner, Bergen (NO)

(21) Appl. No.: 13/911,456

(22) Filed: Jun. 6, 2013

Related U.S. Application Data
(63) Continuation of application No. 12/962,946, filed on Dec. 8, 2010, now abandoned.
(60) Provisional application No. 61/310,109, filed on Mar. 3, 2010.

(51) Int. Cl. C12Q 1/68; G01N 33/68

(52) U.S. Cl. CPC C12Q 1/6886; G01N 33/6893
USPC 506/9; 435/7.1

(57) ABSTRACT

A method for diagnosis of different stages of endometrial cancer and for evaluating the probability of survival for an individual suffering from endometrial carcinoma, and for stratification of a therapy regimen for an endometrial tumor or monitoring therapeutic efficacy in an individual suffering considers the expression status of STMN1 gene or protein. A kit for use in the methods allows for determining amplifications and deletions of chromosomal regions 3q26.32 and 12p12.1, determining alterations of the gene expression profile of the genes (gene signature): upregulation of the genes PLEKHK1, ATP10B, NMU, MMP1, ATAD2, NETO2, TNNI3, PHLDA2, OVOL1 and down-regulation of the genes: NDP, KIAA1434, MME, CFH, MOXD1, SLC47A1, RBP1, PDE8B, ASRGL1, ADAMTS19, EFHD1, ABCA5, NPAS3, SCML1, TNXB, ENTPD3, AMY1A, ENPP, RASL11B, PDZK3, or the expression status of the STMN1 gene or protein.

The present invention relates to a method for diagnosis of different stages of endometrial cancer in an individual. Further, the present invention relates to a method for evaluating the probability of survival for an individual suffering from endometrial carcinoma. In another aspect, the present invention relates to the stratification of therapy regimen of endometrial tumor, ovarian cancer, breast cancer, non-small lung cancer or hormone refractory prostate cancer therapy in an individual or monitoring therapeutic efficacy in an individual suffering from the same based on the expression status of STMN1 gene or protein. Moreover, the present invention relates to a kit for use in any of the above referenced methods comprising a means for determining amplifications and deletions of chromosomal regions 3q26.32 and 12p12.1, determining alterations of the gene expression profile of the genes (gene signature): upregulation of the genes PLEKHK1, ATP10B, NMU, MMP1, ATAD2, NETO2, TNNI3, PHLDA2, OVOL1 and down-regulation of the genes: NDP, KIAA1434, MME, CFH, MOXD1, SLC47A1, RBP1, PDE8B, ASRGL1, ADAMTS19, EFT-1D1, ABCA5, NPAS3, SCML1, TNXB, ENTPD3, AMY1A, ENPP, RASL11B, PDZK3, or the expression status of the STMN1 gene or protein, respectively. Finally, the present invention provides a method for predicting the response to taxanes in an individual suffering from a disease treated with the taxanes based on the expression status of the STMN1 gene or protein.

### PRIOR ART

With a 2-3% lifetime risk among women, endometrial cancer is the most common pelvic gynecologic malignancy in industrialized countries, and the incidence is increasing (Amant F et al. (2005), Lancet, 366:491-505.). Approximately 75% of cases are diagnosed with the tumor confined to the uterine corpus, but 15%-20% of these recur after primary surgery with limited respond to systemic therapy. In light of these recurrences, patients with localized endometrial cancer have 2 major needs: (1) adjuvant therapies that will reduce the recurrence rate, and (2) the ability to target these therapies to the patients most likely to recur. In addition, women with metastatic disease require effective systemic therapy.

These needs, for effective systemic therapies and reliable prognostic markers, have been only partly addressed. The most common basis for determining risk of recurrent disease has been the categorization of endometrial cancer into two subtypes. The majority are type I, associated with good prognosis, low stage and grade, and endometrioid histology. In contrast, type II cancers are characterized by high stage and grade, non-endometrioid histology, and poor prognosis. However, the prognostic value of this distinction is limited as up to 20% of type I cancers recur, while half of type II cancers do not.

The molecular basis of the distinction between type I and II cancer is only partially understood. Type I cancer is associated with hyperestrogenic risk factors, is more often estrogen and progesterone receptor positive, diploid, microsatellite unstable, and KRAS or PTEN mutant. Type II cancer is more often aneuploid and harbors alterations in CDKN2A, TP53, and ERBB2. Such molecular alterations are of prognostic value but have not provided a basis for improved therapy Lax SF, 2004, Virchows Arch, 444:213-223.). Hormone receptor status influences the choice of treatment in metastatic disease, but most aggressive tumors are receptor negative.

Recently, Saal et. al. PNAS, 2007, 104, 18, 7564 to 7569 report on observations that poor prognosis in carcinoma is associated with a gene expression signature of apparent PTEN tumor suppressant pathway activity. That is, expression of STMN1 has been shown previously to correlate with PI3K activity in breast cancer and can be measured by immunohistochemistry in paraffin-embedded tissue.

The present inventors hypothesized that tumors with an aggressive phenotype are likely to be distinguished by underlying genetic alterations reflected in distinct transcriptional signatures, and investigated whether tumors that recur share transcriptional signatures that suggest shared underlying genetic alterations.

Hence, the first object of the present invention is to provide methods allowing diagnosis and differentiation of endometrial carcinoma and other types of carcinoma in an individual. Further, the present invention aims to provide a method of evaluating the probability of survival for an individual suffering from endometrial carcinoma or the clinical outcome thereof as well as providing a method for the stratification of endometrial tumor, ovarian cancer, breast cancer, non-small lung cancer or hormone refractory prostate cancer therapy in an individual or monitoring therapeutic efficacy in an individual suffering therefrom.

### SUMMARY OF THE PRESENT INVENTION

In a first aspect, the present invention relates to a method for diagnosis or differentiation of endometrial carcinoma in an individual comprising the steps of determining alterations, in particular, amplifications and deletions, of chromosomal regions 3(126.32 and 12p12.1, alterations of the gene expression profile of the genes (gene signature): upregulation of the genes PLEKHK1, ATP10B, NMU, MMP1, ATAD2, NETO2, TNNI3, PHLDA2, OVOL1 and down-regulation of the genes: NDP, KIAA1434, MME, CFH, MOXD1, SLC47A1, RBP1, PDE8B, ASRGL1, ADAMTS19, EFHD1, ABCA5, NPAS3, SCML1, TNXB, ENTPD3, AMY1A, ENPP, RASL11B, PDZK3, or the expression status of the STMN1 gene or protein.

In another aspect, the present invention relates to a method for evaluating the probability of survival for individuals suffering from endometrial carcinoma or the clinical outcome comprising the steps of determining amplifications and deletions of chromosomal regions 3q26.32 and 12p12.1, alterations of the gene expression profile of the genes (gene signature): upregulation of the genes PLEKHK1, ATP10B, NMU, MMP1, ATAD2, NETO2, TNNI3, PHLDA2, OVOL1 and down-regulation of the genes: NDP, KIAA1434, MME, CFH, MOXD1, SLC47A1, RBP1, PDE8B, ASRGL1, ADAMTS19, EFHD1, ABCA5, NPAS3, SCML1, TNXB, ENTPD3, AMY1A, ENPP, RASL11B, PDZK3, or the expression status of the STMN1 gene or protein.

In another aspect, the present invention relates to a method for the stratification of endometrial tumor, ovarian cancer, breast cancer, non-small lung cancer or hormone refractory prostate cancer therapy in an individual or monitoring therapy efficacy in an individual suffering from any one of the above identified tumors or cancers comprising the step of determining the special status of the STMN1 gene or protein and stratifying the therapy or monitoring the efficacy of therapy of any one of the referenced tumors or cancers in said individual.

In addition, a method for monitoring the progression of endometrial carcinoma, ovarian cancer, breast cancer, non-small lung cancer or hormone refractory prostate cancer in an individual is provided comprising the steps of
a) detecting the level or amount of STMN1 in a first sample from the individual at a first time point;
b) determining the level or amount of STMN1 in a second sample from the individual at a second point in time; and
c) comparing the level or amount of STMN1 determined in step a) to the level or amount detected in step b) or to a reference value.

Another aspect of the present invention relates to a kit for use in providing a diagnosis or differentiation of endometrial carcinoma in an individual, for the stratification of endometrial tumor therapy in an individual, monitoring therapeutic efficacy in an individual, or for evaluating the probability of survival for an individual suffering from endometrial carcinoma comprising means for determining alterations, in particular, amplifications and deletions, of chromosomal regions 3q26.32 and 12p12.1, alterations of the gene expression profile of the genes (gene signature): upregulation of the genes PLEKHK1, ATP10B, NMU, MMP1, ATAD2, NETO2, TNNI3, PHLDA2, OVOL1 and down-regulation of the genes: NDP, KIAA1434, MME, CFH, MOXD1, SLC47A1, RBP1, PDE8B, ASRGL1, ADAMTS19, EFHD1, ABCA5, NPAS3, SCML1, TNXB, ENTPD3, AMY1A, ENPP, RASL11B, PDZK3, or the expression status of the STMN1 gene or protein.

Finally, the present invention relates to a method for predicting the response or outcome of therapy with taxanes in an individual treated therewith based on the expression status of the STMN1 gene or protein.

The above methods are particularly useful for stratification of the therapy and for monitoring the therapy when treating metastatic cancer, in particular metastatic endometrial cancer.

Finally, the present invention relates to a method for the stratification of therapy or for monitoring the efficacy of the therapy based on PI3K inhibitors, Akt inhibitors, mTOR inhibitors or PTEN activators comprising the step of determining the expression status of the STMN1 gene or protein.

### BRIEF DESCRIPTION OF THE DRAWINGS

FIGS. 1A-1C: Results of unsupervised clustering of expression data from 57 endometrial carcinomas. (A) Two major clusters are identified and recapitulated using the displayed predictor set of 29 genes. Features associated with each tumor are displayed in the bottom 3 panels. PI3K scores in the top textile are called positive. (B) The ability of the 29 predictor genes to distinguish Cluster 1 and 2 tumors was validated in these tumors by quantative RT-PCR. (C) Recurrence-free survival was significantly lower for patients in Cluster 2. Four tumors (all in Cluster 2) were metastatic at presentation and therefore omitted from this analysis.

FIGS. 2A-2B2. Significant copy-number alterations in endometrial cancer. (A) Amplifications (red) and deletions (blue), determined by segmentation analysis of normalized signalintensities from 100K SNP arrays, are displayed across the genome (chromosome positions, indicated along the y axis, are proportional to marker density) for 76 tumors and 9 cell lines. (2B-1,2) GISTIC analysis of copy-number changes. The G-score represents the frequency x average amplitude of the aberrations identified in (A). The FDR q-values, representing the statistical significance associated with these scores (7), are displayed along the bottom. Regions with q-values <0.25 (green lines) were considered significantly altered. The locations of the peak regions of maximal copy-number change, and the known cancer-related genes within those peaks, are indicated to the right of each panel.

FIGS. 3A-3E. Relations between PIK3CA amplification, PI3K activation, and survival. (A) Amplification of a region in 3q26 that includes PIK3CA is significantly associated with poor recurrence-free survival. These amplifications are associated with overexpression of PIK3CA (B) and increased PI3K scores (C). (D) Among the broader set of poor-prognosis tumors in expression Cluster 2, PI3K scores are equally high among tumors without 3q26 amplification as with 3q26 amplification, suggesting alternative methods of pathway activation. (E) Tumors with elevated protein expression of the PI3K pathway member STMN1 had significantly poorer survival, after controlling for age, FIGO stage, histologic subtype, and grade.

FIG. 4. LOH at the PTEN locus is rarely associated with deletions. The top panel displays loss of heterozygosity (LOH) and retention of heterozygosity along chromosome 10 for 8 endometrial carcinomas (labeled A-H) with LOH at the PTEN locus. The bottom panel displays signal intensities (high, low and white=neutral) and copy-number calls (amplified, deleted) for those endometrial carcinomas. LOH at PTEN is associated with deletion only in tumor A. It is associated with amplification in tumor B and neutral copy numbers in tumors C-H. Across our dataset, copy loss is seen in only 4 of 18 endometrial carcinomas observed to have LOH at the PTEN locus.

FIGS. 5A-5C. The signatures of 3q26 amplification and a PI3 kinase inhibitor are associated. (A) Genes with the highest (most over-expressed) and lowest (most under-expressed) t-test statistic in the comparison between 3q26.32 amplified and unamplified samples. (B) Comparison of global expression patterns using the Connectivity Map (1) identifies the PI3 kinase inhibitor LY-294002 as the most significantly anticorrelated with the 3q26 amplification signature among the 164 small molecules tested. Each of the N instances in which the small molecule was tested in the Connectivity Map was scored according to this signature. The p-value for each small molecule represents the distribution of these scores compared to the distribution of scores among all small molecules, using a permutation test as described in Lamb et al, Science 2006 Lamb J et al. (2006) (The Connectivity Map: using gene-expression signatures to connect small molecules, genes, and disease. Science 313:1929-1935). (C) The rankings of the 3q26 amplification signature scores for the 17 instances of LY-294002 are displayed as black bars. Rankings within the green and red regions represent scores that are respectively aligned with and divergent from the 3q26 amplification signature.

FIGS. 6A-6G. Endometrial carcinomas with 3q26-27 amplification tend to overexpress PIK3CA and have high PI3K scores and aggressive features. (A) Functional amplification scores for 3q26-27 are significantly higher among samples with 3q26.32 amplification in the primary investigation set. Functional amplification scores were generated from genome-wide expression data as previously described (Carter SL et al. (2006) A signature of chromosomal instability inferred from gene expression profiles predicts clinical outcome in multiple human cancers. Nat Genet 38:1043-1048). Amplification of 3q26.32 was determined by SNP array analysis. The finding that the two correlate validates the use of these functional amplification scores as a measure of 3q26-27 amplification. (B) Expression of PIK3CA and (C) PI3K scores are higher among tumors in the validation set with high 3q26-27 functional amplification scores. The vertical lines represent the minimum threshold of functional amplification score typically seen in 3q26.32 amplified tumors in (A). The box plot insets represent PIK3CA expression levels and PI3K scores in samples with functional amplification scores lower and higher than this threshold, representing tumors likely to be unamplified and amplified respectively at the PIK3CA locus. P-values corresponds to a test of the null-hypothesis that the Pearson product-moment correlation was equal to 0. High 3q26-27 functional amplification scores are also associated with (D) high grade and (E) non-endometrioid histology, as are (F-G) high 29-gene summary scores, representing membership in expression Cluster 2. These comparisons were made in the validation dataset. P-values were calculated using a one-sided Wilcoxon test.

FIGS. 7A-7G. Association between measures of PI3 kinase activation and genomic and proteomic features of endometrial cancer. (A) Using the Connectivity Map (1) the anticorrelation between the Cluster 2 expression signature and the PI3 kinase inhibitor LY-294002 was the third-strongest among the 164 small molecules tested. (B) The rankings of the Cluster 2 signature scores for the individual instances of LY-294002 in the Connectivity Map are displayed as in SI FIG. 2C. (C) PI3K scores, an alternative measure of PI3 kinase pathway activation, are significantly associated with decreased recurrence-free survival. (D) PI3K scores are not significantly associated with PTEN mutations or (E) PIK3CA mutations. P-values were calculated using a two-sided Wilcoxon test. (F) High expression of the PI3 kinase pathway member STMN1, seen by immunohistochemistry in a tumor with amplification of PIK3CA and elevated expression of PIK3CA and the PI3 kinase activation signature. (G) Low expression of STMN1, seen in a tumor without amplification of PIK3CA or elevated expression of PIK3CA or the PI3 kinase activation signature.

### DETAILED DESCRIPTION OF THE PRESENT INVENTION

In a first aspect, the present invention relates to a method for the diagnosis or differentiation of endometrial carcinoma in an individual comprising the step of determining alterations, in particular, amplifications and deletions, of chromosomal regions 3q26.32 and 12p12.1, alterations of the gene expression profile of the genes (gene signature): upregulation of the genes PLEKHK1, ATP10B, NMU, MMP1, ATAD2, NETO2, TNNI3, PHLDA2, OVOL1 and down-regulation of the genes: NDP, KIAA1434, MME, CFH, MOXD1, SLC47A1, RBP1, PDE8B, ASRGL1, ADAMTS19, EFHD1, ABCA5, NPAS3, SCML1, TNXB, ENTPD3, AMYI A, ENPP, RASL11B, PDZK3, or the expression status of the STMN1 gene or protein.

That is, the present inventors recognized that two major groups of tumors can be distinguished in patients suffering from endometrial carcinoma. Namely, two clusters allow to differentiate between two major groups of tumors whereby these clusters identify a two-fold or higher change for 138 significant genes of which 64 where upregulated and 74 downregulated in cluster 2. A set of 29 genes, validated by quantitative RT-PCR, predicted the clusters with 100% accuracy.

The expression clusters identified herein have strikingly different clinical and histopathologic characteristics. Cluster 2 contained more aggressive tumors containing almost all type II tumors. In addition, patients with tumors in Cluster 2 had significantly poorer recurrence-free survival. Segregation into Cluster 2 predicted recurrence better than known means in the art, like International Federation of Gynecology and Obstetrics (FIGO) stage, histologic grade, number of mitosis, presence of a non-endometrioid histologic subtype, tumor necrosis and vascular invasion. of the gene expression profile of the genes (gene signature): upregulation of the genes PLEKHK1, ATP10B, NMU, MMP1, ATAD2, NETO2, TNNI3, PHLDA2, OVOL1 and down-regulation of the genes: NDP, KIAA1434, MME, CFH, MOXD1, SLC47A1, RBP1, PDE8B, ASRGL1, ADAMTS19, EFHD1, ABCA5, NPAS3, SCML1, TNXB, ENTPD3, AMY1A, ENPP, RASLIIB, PDZK3, as well as determining the expression status of STMN 1 gene or protein in an individual in vivo or in vitro allows for the diagnosis or differentiation of endometrial carcinoma in said individual.

According to the present invention, the methods disclosed herein relates to in vitro 10 and/or in vivo methods, respectively.

In a preferred embodiment, the method for diagnosing or differentiation of endometrial carcinoma in an individual comprise the steps of determining the PI3K activity in patients having aggressive endometrial carcinoma, in particular, based on the alterations in 3q26.32 or on the expression status of STMN1 gene or protein.

In another embodiment, it is preferred that the expression status of the STMN1 gene or protein is determined.

The method of the present invention allows to differentiate between high grade aggressive phenotype of endometrial cancer and low grade phenotype of endometrial cancer.

Hence, present invention relates to methods allowing diagnosis and differentiation of endometrial carcinoma, in particular allowing to differentiate between low grade and high grade aggressive phenotype in endometrial carcinoma based on the STMN1 expression. The present invention is directed to the diagnosis, prognosis as well as to the stratification of endometrial tumors and its therapy. That is, in one aspect, the present invention relates to a method for diagnosing and/or identifying endometrial carcinoma and the importance of the PI3K pathway in patients having aggressive endometrial cancer. The STMN1 expression correlates with PI3K scores and, in addition, high STMN1 expression is associated with poor recurrence free survival and with poor recurrence free and overall survival in patients suffering from endometrial carcinomas. It is demonstrated herein that high STMN1 expression represents an independent prognostic indicator allowing to differentiate between high grade aggressive phenotype and low grade phenotype of endometrial cancer. In particular, high STMN1 expression is associated with poor prognosis and the otherwise low risk endometrioid subgroup.

The present inventors recognized that PI3K activity associates with poor prognosis, thus, indicating that measuring PI3K activity allows to improve prognostication of localized endometrial cancer.

The present invention covers the determination of STMN1 expression in methods allowing the diagnosis and differentiation of endometrial carcinoma as well as stratification of endometrial tumors and its therapy as well as monitoring the therapy.

Furthermore, the present invention provides a method for evaluating the probability of survival as well as methods for providing a prognosis of a subject afflicted with endometrial cancer based on PI3K activity and/or STMN1 expression.

In further aspects, the present invention relates to methods including determining amplifications and deletions of specific chromosomal regions, like 3q and 12p, in particular 3q26.32 and 12p12.1 as detailed herein. In particular, the amplifications and deletions outlined in FIG. 2 allows to differentiate individuals afflicted with endometrial carcinoma in two clusters, namely cluster 1 and cluster 2 having significant differences in disease-free survival. Preferably, the methods according to the present invention includes determining expression of STMN1 in combination with determining at least one of the amplifications or deletions in the chromosomal regions identified herein or determining the gene signature of the genes PLEKHK1, ATP10B, NMU, MMP1, ATAD2, NETO2, TNNI3, PHLDA2, OVOL1 and NDP, KIAA1434, MME, CFH, MOXD1, SLC47A1, RBP1, PDE8B, ASRGL1, ADAMTS19, EFHD1, ABCA5, NPAS3, SCML1, TNXB, ENTPD3, AMY1A, ENPP, RASL11B, PDZK3.

To conclude, the present invention relates in another aspect to a method for evaluating the probability of survival for a patient with endometrial cancer, said method being characterized in that it comprises measuring the level or expression of STMN1 on nucleic acid or amino acid level in a sample obtained from said patient.

Moreover, in another preferred embodiment, the method according to the present invention comprises determining the expression status of STMN1. It has been recognized that high STMN1 expression is associated with poor recurrence-free survival and over survival in patients suffering from endometrial carcinoma. In particular, the STMN1 expression allows to differentiate between high grade aggressive phenotype and low grade phenotype of endometrial carcinoma whereby high STMN1 is associated with high grade aggressive phenotype of endometrial carcinoma.

In another preferred embodiment, the methods according to the present invention comprises the step of determining expression of the STMN1 gene in combination with determining alterations, in particular, the amplifications or deletions, in the chromosomal regions 3q 26.32 and 12p12.1, or altered expression of the gene signature of the genes: PLEKHK1, ATP10B, NMU, MMP1, ATAD2, NETO2, TNNI3, PHLDA2, OVOL1 (upregulation) and of the genes: NDP, KIAA1434, MME, CFH, MOXD1, SLC47A1, RBP1, PDE8B, ASRGL1, ADAMTS19, EFHD1, ABCAS, NPAS3, SCML1, TNXB, ENTPD3, AMY1A, ENPP, RASL11B, PDZK3 (downregulation).

In another aspect, the present invention relates to a method for providing a prognosis of a subject with an endometrial carcinoma disorder comprising the steps of:
a) obtaining a sample from said subject
b) determining the expression status of the gene STMN1 in said sample
c) determining therefrom the prognosis of said subject whereby over said expression is indicative of negative prognosis.

In a further aspect, the present invention relates to a method for diagnosing or identifying endometrial cancer with high grade aggressive phenotype in a subject comprising
a) determining the level or amount of STMN1 in a sample of said subject; and
b) comparing the level or amount determined in step a) to a reference value, wherein an increase in the level or amount relative to the reference value is indicative for endometrial carcinoma with high grade aggressive phenotype.

Another aspect relates to a method for the stratification of the therapeutic regimen of a subject with endometrial carcinoma comprising
a) determining the level or amount of STMN1 in a sample of said subject; and
b) determining the therapeutic regimen based on the level or amount of STMN1.

Further, the present invention relates to a method for predicting a clinical outcome or determining the treatment caused in a subject afflicted with endometrial carcinoma, comprising:
a) determining the level or amount of STMN1 in at least one sample of said subject; and
b) predicting clinical outcome or determining the treatment course based on the amount or level of STMN1 present in said sample.

In addition, the present invention relates to a method for monitoring the progression of endometrial carcinoma in a subject, comprising or detecting the level or amount of STMN1 in a first sample from the subject at a first point in time;
b) determining the level or amount of STMN1 in a second sample from the subject at a second point in time; and
c) comparing the level or amount of STMN1 determining as step a) to the level or amount detected in step b) or to a reference value.

Moreover, the present invention relates to a method for the stratification of endometrial tumor, ovarian cancer, breast cancer, non-small lung cancer or hormone refractory prostate cancer therapy in an individual or monitoring therapeutic efficacy of said types of cancer in an individual comprising the step of determining the expression status of the STMN1 gene and stratifying the therapy or monitoring the efficacy of therapy of the endometrial tumor, ovarian cancer, breast cancer, non-small lung cancer or hormone refractory prostate cancer in said individual.

That is, the present inventors recognized that not only for the stratification of endometrial cancer and for monitoring therapeutic efficacy in the treatment of endometrial tumors and cancers but also in ovarian cancer, breast cancer, non-small lung cancer or hormone refractory prostate cancer therapy, STMN1 is a valuable biomarker.

In particular, the present inventors aimed in demonstrating that STMN1, also known as Stathmin, expression predicts the response to taxanes in metastatic endometrial cancer. Hence, Stathmin expression is useful as a marker for the treatment of metastatic endometrial cancer but also in endometrial cancer in general and ovarian cancer, breast cancer, non-small lung cancer or hormone refractory prostate cancer.

The method for the stratification of the therapeutic regimen or monitoring the therapeutic regimen or monitoring the therapeutic efficacy of an individual suffering from endometrial cancer, ovarian cancer, breast cancer, non-small lung cancer or hormone refractory prostate cancer comprises the step of determining the level or amount of STMN1 is a sample of said individual and determining the therapeutic regimen or strategy or monitoring the therapeutic efficacy based on the level or amount of STMN1.

Preferably, the STMN1 expression status is determined on nucleic acid or amino acid level in said individual.

The skilled person is well aware of suitable methods for determining the expression status of the gene STMN1 or the amplification and deletions in the chromosomal regions 3q 26.32 and 12p12.1, as well of determining alterations of the genes PLEKHK1, ATP10B, NMU, MMP1, ATAD2, NETO2, TNNI3, PHLDA2, OVOL1 (upregulation) and of the genes: NDP, KIAA1434, MME, CFH, MOXD1, SLC47A1, RBP1, PDE8B, ASRGL1, ADAMTS19, EFHD1, ABCA5, NPAS3, SCML1, TNXB, ENTPD3, AMY1A, ENPP, RASL11B, PDZK3 (downregulation), respectively.

Preferred embodiments include the detection of nucleic acid level using PCR methods or hybridisation methods using suitable marker molecules.

On protein level, determining the expressionstatus of the gene STMNI may be effected by using appropriate antibodies and systems comprising the same. Suitable methods including ELISA, Western blot, immunohistochemical or immunofluorescence detection.

In another aspect, a kit for use in providing a diagnosis or differentiation of endometrial carcinoma in an individual, for the stratification of endometrial tumor therapy in an individual, monitoring therapeutic efficacy in an individual, or for evaluating the probability of survival for an individual suffering from endometrial carcinoma comprising means for determining amplifications and deletions of chromosomal regions 3q26.32 and 12p12.1, the expression status of the STMN1 gene or protein or means for determining amplification and deletions whereby said amplifications (upregulation) and deletions (downregulations) are amplifications of the genes: PLEKHK1, P10B, NMU, MMP1, ATAD2, NETO2, TNNI3, PHLDA2, OVOL1 and deletions of the genes: NDP, KIAA1434, MME, CFH, MOXD1, SLC47A1, RBP1, PDE8B, ASRGL1, ADAMTS19, EFHD1, ABCA5, NPAS3, SCML1, TNXB, ENTPD3, AMY1A, ENPP, RASL11B, PDZK3 is provided.

In another aspect, said kit comprises means for determining the PI3K activity in patients having aggressive endometrial carcinoma.

Particularly preferred, said kit according to the present invention is suitable for providing diagnosis or differentiation of endometrial carcinoma in an individual or for the stratification of the therapeutic regiment of monitoring the therapeutic efficacy comprising means for detecting STMN1 expression status.

Said kit is particularly useful for predicting the response to taxanes in an individual when treating the same considering a therapeutic regimen using taxanes in said individuals. In particular in case of the treatment of metastatic endometrial cancer, the method and kits according to the present invention are useful for stratifying the therapy thereof. For example, when taxanes are used for the treatment of metastatic cancer, like metastatic endometrial cancer, determining the STMN1 status allows to stratify and to diagnostic therapeutic success of taxanes treatment.

That is, there are a few markers available to predict response to treatment of metastatic endometrial cancer. Patients with tumors expressing estrogen and progestagen receptors have the best response to antihormonal treatment. However, more markers are needed to predict the response to other therapy modalities in patients with metastatic endometrial cancer. It has been demonstrating herein that the level of stathmin expression (STMN1 expression) allows to predict response to tumuli stabilizing chemotherapy in cancer, like endometrial cancer. A typical example of tubuli stabilizing therapy includes Taxol, Taxotere, Eleutherobin, Sarcodicytin A, Sarcodicytin B, Epothilone A, Epothilone B, Discodermolide, Laulimalide, Isolaulimalide, Ixabepilone, Vinblastin, Vinkristin, Vinorelbin.

Finally, the present invention relates to a method for stratification of endometrial tumor or endometrial cancer, ovarian cancer, breast cancer, non-small lung cancer or hormone refractory prostate cancer therapy in an individual or monitoring therapeutic efficacy in an individual whereby the therapy, in particular, the endometrial tumor therapy based on PI3K inhibitors AKT inhibitors or mTOR inhibitors or PTEN activators comprising the step of determining the expression status of the STMN1 gene or protein and stratifying the therapy or monitoring the efficacy of the therapy accordingly.

### Materials and Methods

### Patient Series

For the primary investigation series, primary endometrial carcinomas were immediately frozen during hysterectomies conducted from 2001-2003. All samples were reviewed by a pathologist according to published criteria (Scully RE et al. (1994) Histological typing of female genital tract tumours. International histological classification of tumours. World Health Organization. Springer-Verlag, Berlin Heidelberg). Treatment included bilateral salpingo-oophorectomy and pelvic lymphadenectomy. Adjuvant therapy was recommended for patients with FIGO surgical stage JIB or higher disease or non-endometrioid histology. Patients were followed from primary surgery until June 2007 or death, with a median follow-up for survivors of 3.6 years (range 0.8-5.5). Deaths not attributable to endometrial cancer were censored. No patient was lost to follow-up.

### RNA Analysis

RNA was extracted from biopsies with at least 50% (usually >80%) tumor content using the RNeasy kit (Qiagen). Quality and yield were assessed by agarose electrophoresis, the Agilent Bioanalyser 2100, and spectrophotometry. RNA was prepared in 2 batches and hybridized to Agilent 21K and 22K arrays respectively, according to manufacturer's instructions (www.agilent.com). Arrays were scanned using the Agilent Microarray Scanner Bundle.

Signal intensities were determined using J-Express (www.molmine.com) and filtered to remove genes with signal intensities below 2 standard deviations over background in either channel (Cy5, Cy3) in more than 30% of samples. Batch adjustment was performed as previously described (Engelsen IB et al. (2008) Br J Cancer 98:1662-1669). Genes were mean-centered across the tumor set.

Hierarchical clustering was performed using the 3500 genes with highest variance using weighted average linkage (WPGMA) and Pearson correlation as similarity measures. Clustering with more or fewer genes gave stable results (data not shown). A SAM analysis using these clusters as class labels identified 138 significantly changed genes, of which 29 were selected for their combined discriminatory power as described in SI Methods. Messenger RNA levels for these 29 genes and PTEN were validated by quantitative PCR using the TaqMan Low Density Array (Applied Biosystems) according to manufacturer's instructions (Engelsen IB et al. (2008) Br J Cancer 98:1662-1669).

For the external dataset (Affymetrix U133+2 arrays), individual probes were sequence-matched against Aceview (NCBI35) (Carter SL et al. (2006) Nat Genet 38:1043-1048) to construct transcript-level probesets. Summary expression levels were then derived by batch-normalization across samples via RMA (Irizarry RA et al. (2003) Nucleic Acids Res 31:e15).

The PI3K score was obtained by comparing previously published expression data of 9 replicate transfections of activated PIK3CA to 5 GFP controls, and includes the 495 genes surpassing a Bonferroni-corrected 2-sided t-test p-value of 0.05. To evaluate this signature, expression data for each gene were normalized to a common mean and scaled to the same standard deviation. For each sample, the activation score is the sum of genes significantly upregulated in the cells with activated PIK3CA (relative to the cells with GFP control) minus genes significantly downregulated in those cells.

### DNA Analysis

Genomic DNA was extracted from surgically dissected, fresh-frozen primary tumors and from nine cell lines: Ishicawa, HeclA, KLE, AN3-CA, EFE184, MFE-280, MFE296, MFE-319, RL-95-2. Tumors were needle dissected to ensure 80% purity.

PIK3CA, KRAS and PTEN were sequenced. Genomic DNA was analyzed by SNP arrays interrogating 116,204 SNP loci (Affymetrix) and the GISTIC algorithm, as previously described in Beroukhim R et al. (2007) Proc Natl Acad Sci USA 104:20007-20012. SNP, gene, and cytogenetic band locations are based on the hg16 (July 2003) genome build (genome.ucse.edu).

### Statistics

For relations of molecular data to clinical phenotype, Pearson's chi-square- (x²), Fisher's exact-, Mann-Whitney-, or Kruskal-Wallis tests were used as appropriate. P-values represent 2-sided tests except when testing the 1-sided hypothesis that 3q^{am}P correlates with measures of PI3K activation. Univariate survival analyses were performed by the Kaplan-Meier method. The log-rank (Mantel-Cox) test with Bonferroni correction was used to compare survival curves for different categories. Variables with significant impact on survival (p<0.05) were further examined by log-minus-log plot before incorporation in the Cox' proportional hazards regression model.

### Results and Discussion

Unsupervised analysis of expression profiles distinguishes aggressive tumors Genome-wide expression and clinical and histopathologic data from a random sampling of 57 endometrial carcinomas in a population-based tissue bank of gynaecologic cancer in Hordaland County, Norway, were collected. The characteristics of these patients were not significantly different from all patients diagnosed with endometrial carcinoma in a ten-year period from the same region, see Table 1.

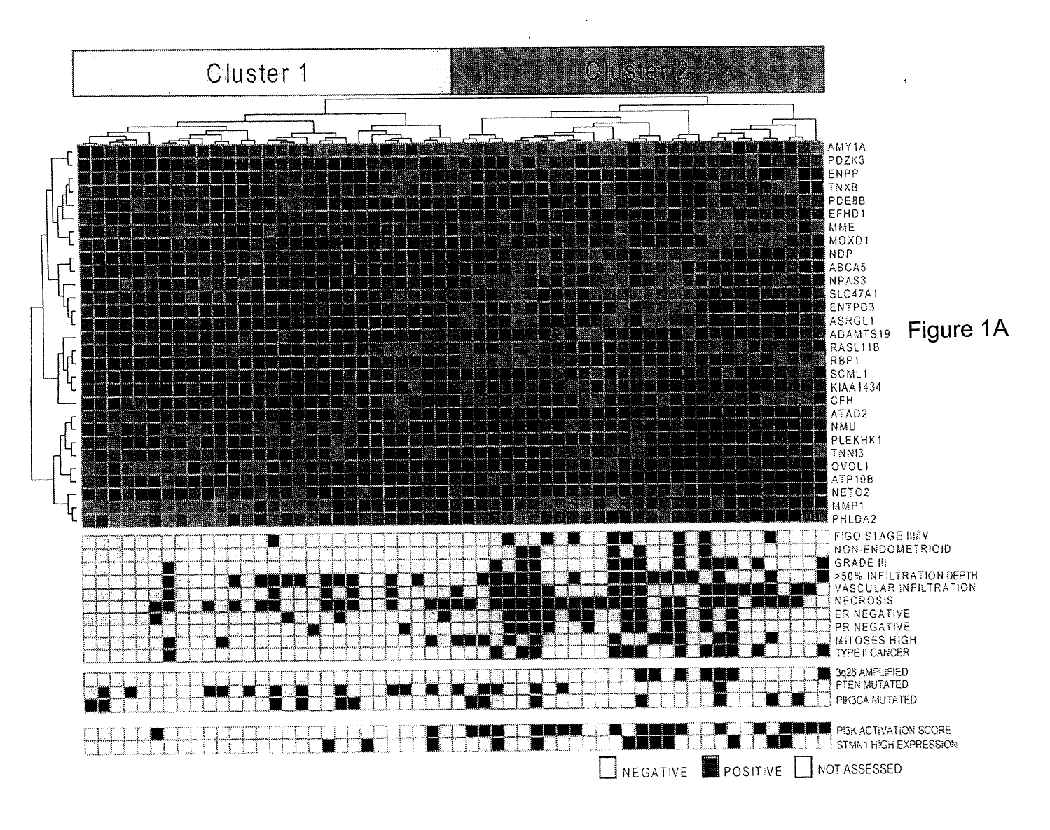

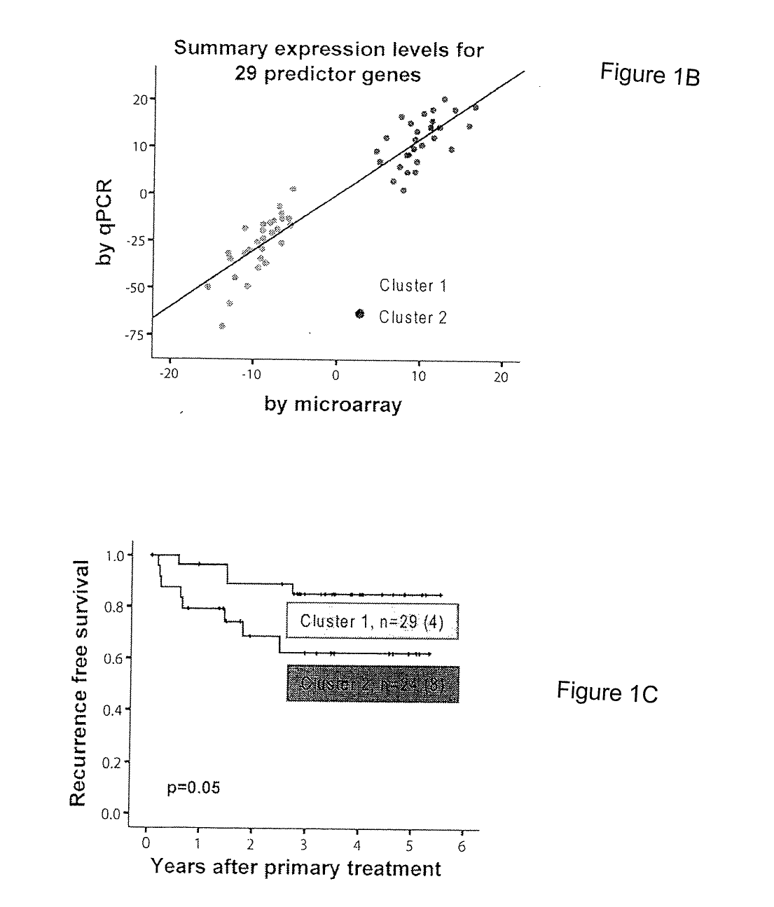

An unsupervised analysis of these data distinguished two major groups of tumors (Clusters 1 and 2, FIG. 1A). SAM analysis (Tusher V G, Tibshirani, R & Chu G (2001) Proc Natl Acad Sci USA 98:5116-5121) between these clusters identified a two-fold or higher change for 138 significant genes, of which 64 were upregulated and 74 downregulated in Cluster 2 (data not shown). A set of 29 genes, validated by quantitative RT-PCR, predicted the clusters with 100% accuracy (FIG. 1A-B).

The two clusters had strikingly different clinical and histopathologic characteristics. Cluster 2 contained more aggressive tumors, with higher International Federation of Gynecology and Obstetrics (FIGO) stage, histologic grade, number of mitoses, presence of non-endometrioid histologic subtype, tumor necrosis and vascular invasion, (p<0.001 for presence of any of these; FIG. 1A, Table 2). Cluster 2 contains almost all the type II tumors (p<0.001) (FIG. 1A, Table 2), but it also contains almost one-third of the type I tumors, and these have more vascular invasion, necrosis, and frequent mitoses than the type I tumors in Cluster 1 (p=0.01). The 29-gene summary set was also significantly correlated with aggressive cancer (Table 2).

Most prominently, patients with tumors in Cluster 2 had significantly poorer recurrence-free survival (p=0.05, FIG. 1C). Segregation into Cluster 2 predicted recurrence better than FIGO stage, histologic subtype, or receptor status, and slightly poorer than grade (Table 3), but did not exhibit independent prognostic impact, most likely due to the limited number of cases and events.

Regions of significant amplification, deletion, and LOH

To identify the underlying somatic changes distinguishing aggressive tumors with the Cluster 2 signature, a genome-wide survey of copy-number changes and LOH among 84 tumors was performed. The majority exhibit a small number of amplifications (median of 4 in each tumor) and even fewer deletions (median of 1). Nevertheless, virtually every region of the genome is amplified or deleted in at least 1 tumor (FIG. 2A).

To distinguish copy-number changes associated with endometrial cancer from potentially random events, we applied the statistical method Genomic Identification of Significant Targets In Cancer (GISTIC) (Beroukhim R et al. (2007) Proc Natl Acad Sci USA 104:20007-20012). GISTIC assigns each region of the genome 2 G-scores (FIG. 2B), each representing the combined frequency and amplitude of either local amplifications or deletions. It then compares these to similar scores generated from random permutations of the data to determine False Discovery Rate q-values, representing the likelihood of obtaining the observed G-scores from chance events alone. The G-scores tend to be larger for amplifications than deletions (FIG. 2B) due to the greater prevalence of amplifications (FIG. 2A). Conversely, deletions attain statistical significance (using a q-value threshold of 0.25, green lines in FIG. 2B) at lower prevalence due to their overall infrequency.

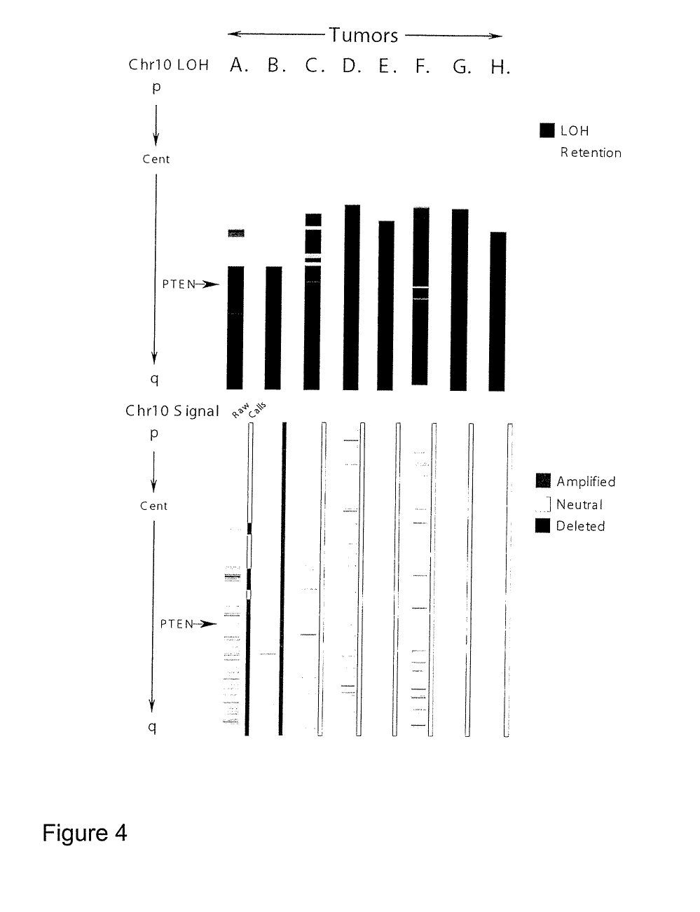

11 significantly amplified and 13 significantly deleted regions of the genome (Table 4A) have been found. For each we selected the peak region, with the highest frequency and amplitude of events, as the region most likely to contain a cancer gene target was selected. Known oncogenes are located within these peaks for 8 amplified regions and known tumor suppressors are located within deletion peaks on chromosomes 1 and 3 (Table 4A), but functional data tying any of these genes to endometrial carcinogenesis are lacking. Also, 14 regions contain no known cancer genes. These usually represent infrequent events (<17% of tumors), with the exception of 1q amplification, where the gene target is unclear due to the large size of the amplicon. The consistent breadth of this amplicon, in fact, may suggest more than one target. LOH generally reflects deletions, with the exception of prevalent copy-neutral LOH on 10q (FIG. 4) containing the known endometrial tumor suppressor PTEN.

Amplifications of KRAS and PIK3CA Associate with Poor Prognosis

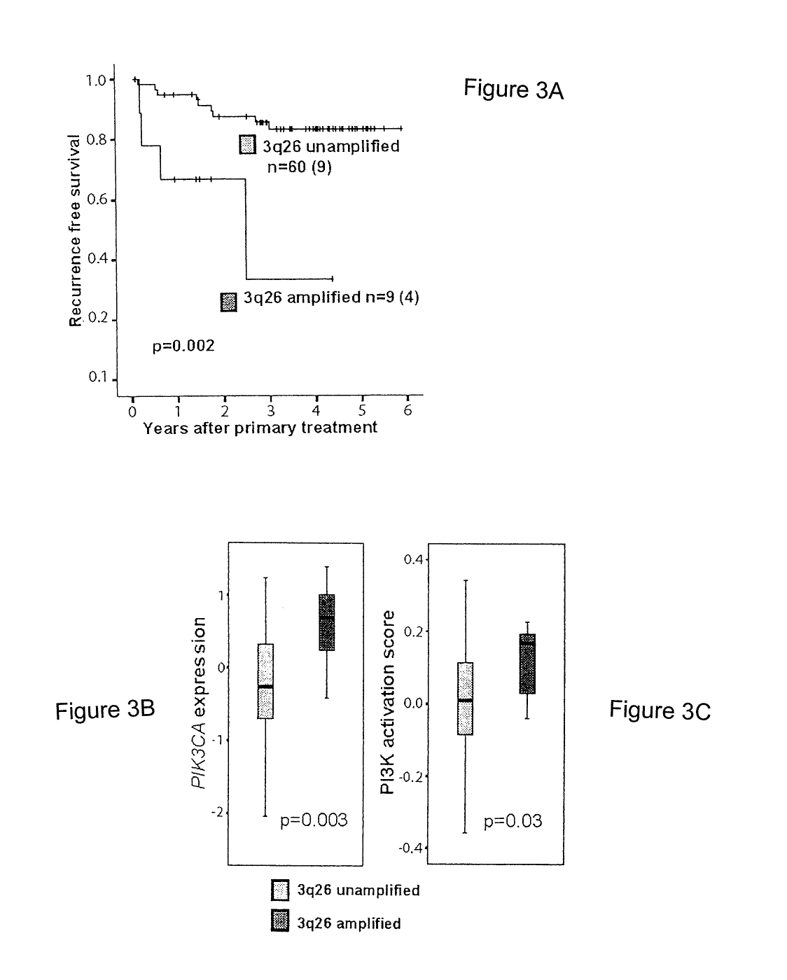

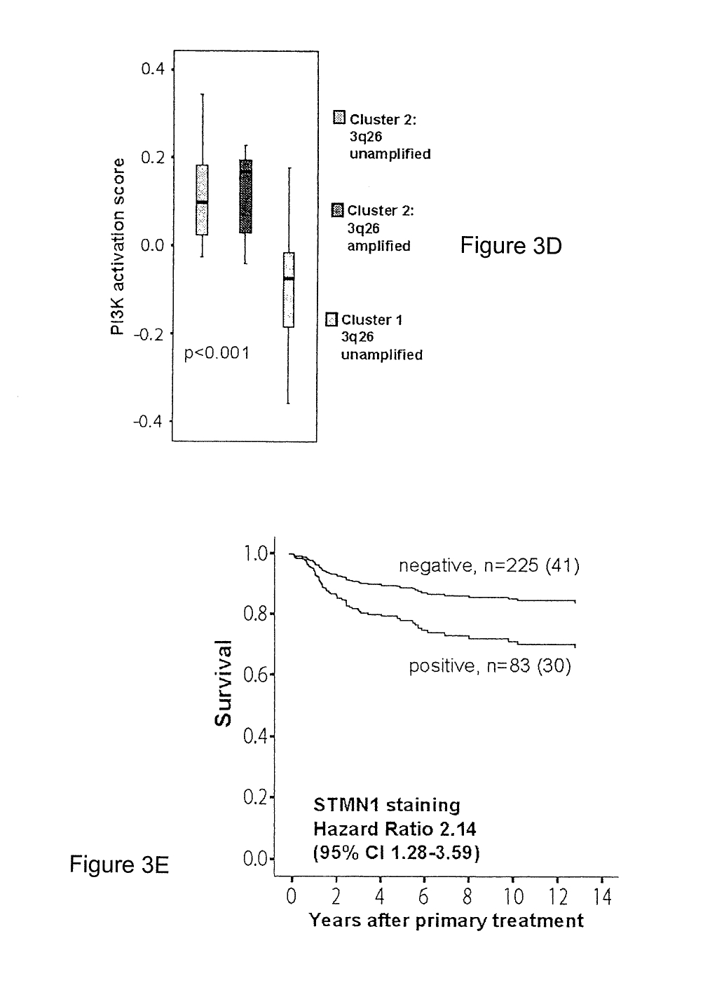

Among the 11 significant amplifications, only 2 (3q26.32 and 12p12.1) are associated with recurrence-free survival (in both cases poor survival) after correction for multiple hypotheses (Table 4B, FIG. 3A). The amplifications due to the low prevalence of deletions were considered only. Amplification of 3q26.32 (3q^{am}P) is also associated with non-endometrioid histology (44% vs 11% prevalence; p=0.02) and high grade (p<0.001). The association between 12p12.1 amplification and poor survival is surprising because mutations of KRAS, which is within the peak region, are known to associate with better survival. However, KRAS in 64 tumors were sequenced and found none of the 12p12.1 amplified samples had mutant KRAS, although mutations were seen in 4 unamplified samples. Amplification of 12p12.1 is also associated with high grade (p=0.02) and FIGO stage (p=0.04). Although 3q26.32 and 12p12.1 tended to be amplified in the same tumors (p=0.03), they usually did not coincide. We directed further analyses at 3q^{am}P because all the samples with this amplification segregated into expression Cluster 2 (p=0.01, FIG. 1A), suggesting that the amplification could be associated with the Cluster 2 transcriptional profile (see below).

Integrated Analyses Associate Markers of PI3 Kinase Activation with Aggressive Cancer

It should be investigated whether 3q^{amP} leads to an aggressive phenotype through activation of PIK3CA. Although PIK3CA has not been shown to be the 3q^{am}P target, its suspected for four reasons: (1) PIK3CA is 1 of 36 genes within the peak region; (2) tumors with 3q^{am}P overexpress PIK3CA compared to unamplified tumors (p=0.003, FIG. 3B); (3) similar amplifications in ovarian cancer act through PIK3CA Shayesteh L et al. (1999) Nat Genet 21:99-102; and (4) the PI3 kinase (PI3K) pathway is frequently aberrant in endometrial cancer, including point mutations in PIK3CA.

Therefore it has been looked for wider effects of PIK3CA activation in the transcriptome of tumors with 3q^{amP}. Published data (Potti A et al. (2006) Nat Med 12:1294-1300) from cell lines transfected with mutationally activated PIK3CA have been used to define a PI3K activation score (PI3K score), representing the expression levels of genes that correlate with activated PIK3CA (see Methods). Tumors with 3q^{am}P scored higher than unamplified samples (p=0.05; FIG. 3C). However, the impact of this finding is limited by its borderline statistical significance and by the possibility that the PI3K score may not reflect PI3K activation generally, but only in the model systems in which it was measured.

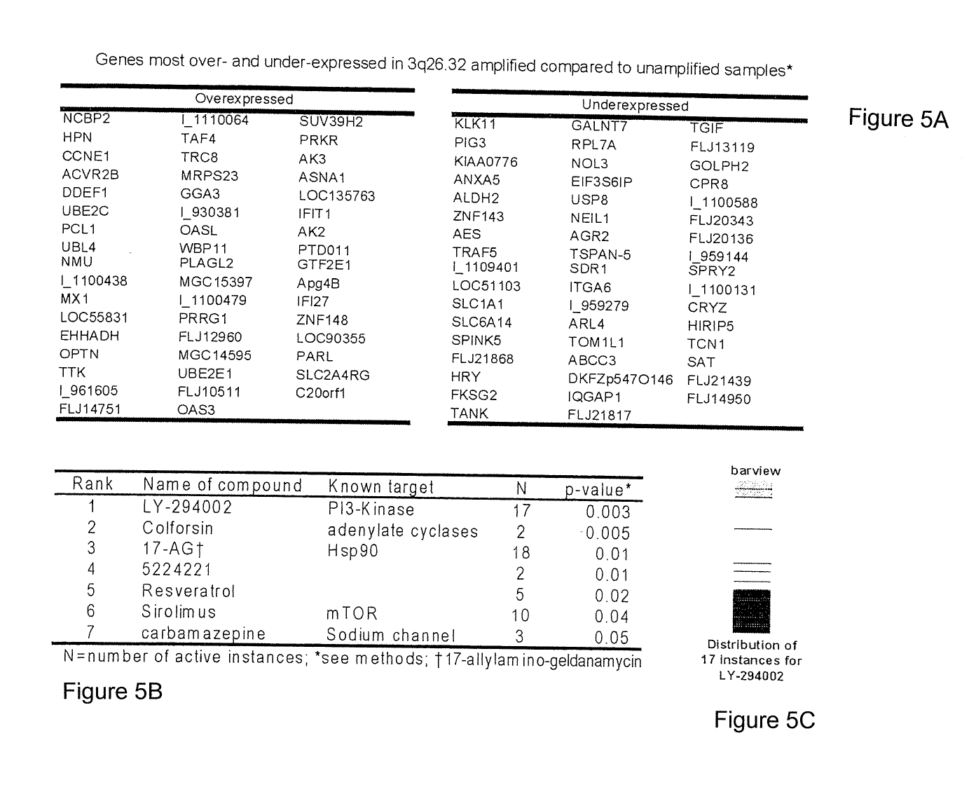

To corroborate this finding it has been analyzed whether samples with 3qamP have an expression profile opposite that induced by PI3K pathway inhibition. To that end, the 50 most overexpressed and underexpressed genes in samples with 3q^{a}mP relative to unamplified samples (FIG. 5A) have been queried using the Connectivity Map (Lamb J et al. (2006) Science 313:1929-1935). Among 164 small molecules represented in the Connectivity Map, the PI3K inhibitor LY-294002 (Vlahos g et al. (1994) J Biol Chem 269:5241-5248) had an expression signature most significantly anticorrelated with the 3q^{am}P signature (SI FIG. 2B-C, p=0.003). LY-294002 is known to bind to additional kinases, raising the possibility that this anticorrelation is due to non-specific effects. The anticorrelation between the 3q^{am}P signature and inhibitors of adenylate cyclase and Hsp90 (FIG. 5B) also suggests potentially complex effects of the amplicon. Nevertheless, the findings that the 3q^{am}P signature correlates with a PI3K activation signature and anticorrelates with the signature of a PI3K inhibitor support the hypothesis that one of the effects of 3q^{am}P may be to increase PI3K activity.

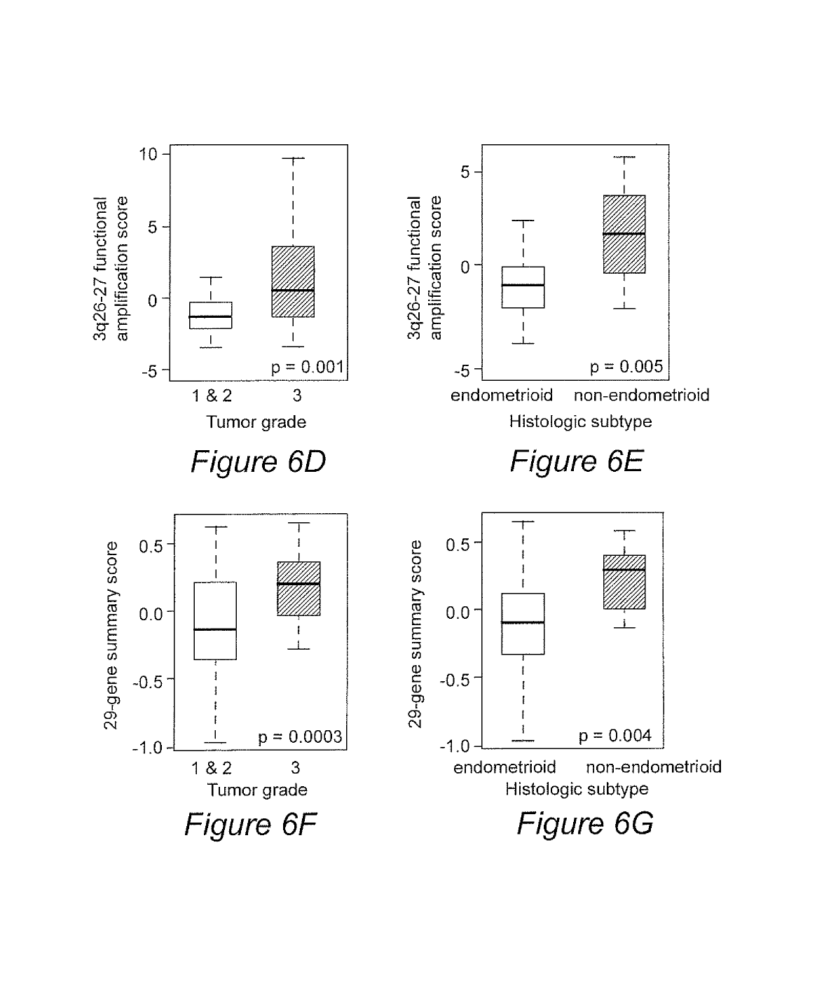

Further, the correlation between PIK3CA amplification and the PI3K score in an independent expression dataset has been validated. First, amplification of 3q26-27 from local gene expression levels has been inferred, as reflected in a ‘functional amplification’ (FA) score. As expected, samples determined to have 3crP by SNP array analysis also had high 3q26-27 FA scores (p<10⁻⁵, FIG. 6A), confirming the score as a meaningful assessment of amplification status. We then inferred 3q26-27 amplification levels in a publicly available expression dataset of 134 endometrial tumors (http://expo.intgen.org/geo/home.do) The correlations between 3q^{am}P and both PIK3CA overexpression and the PI3K score validated (p=2×10^{-1°} and 7×10⁻⁵, respectively; FIG. 6B-C).

In addition, the correlations between aggressive phenotype and both PIK3CA amplification and the Cluster 2 signature in this independent dataset has been validated. Although survival data were unavailable, both available markers of poor survival, high grade and non-endometrioid subtype, correlated with high 3q26-27 FA scores (p=0.001 and 0.005, respectively; FIG. 6D-E) and with high values of the 29 gene summary predictor for membership in Cluster 2 (p=3×10⁻⁴ and 0.004, respectively; FIG. 6F-G).

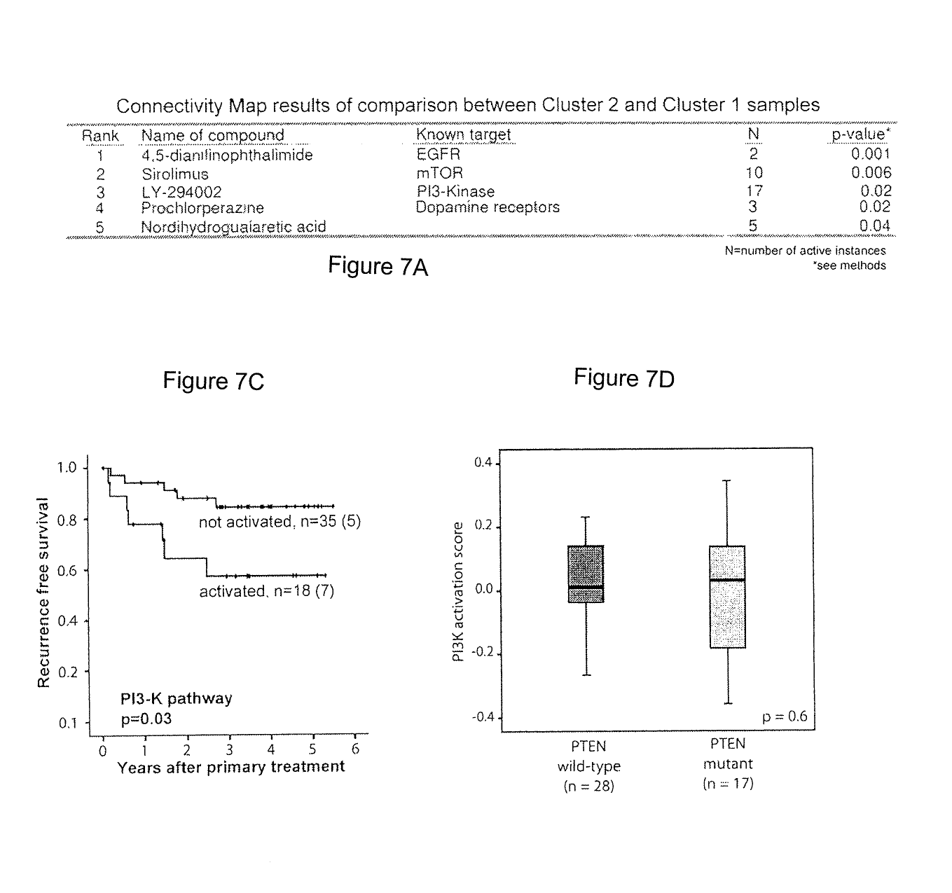

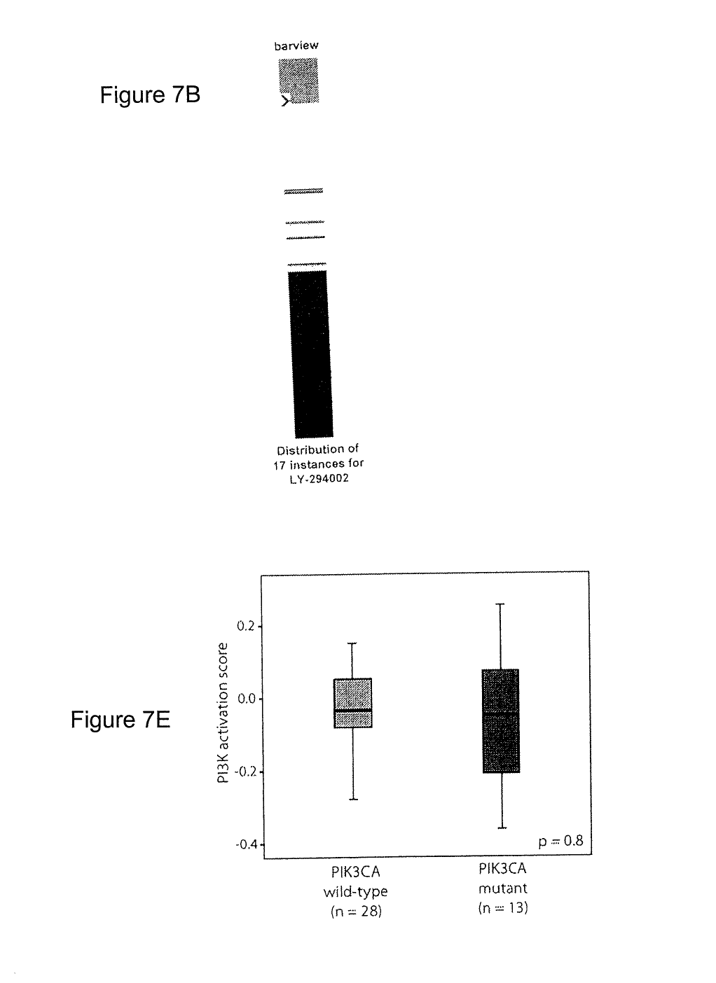

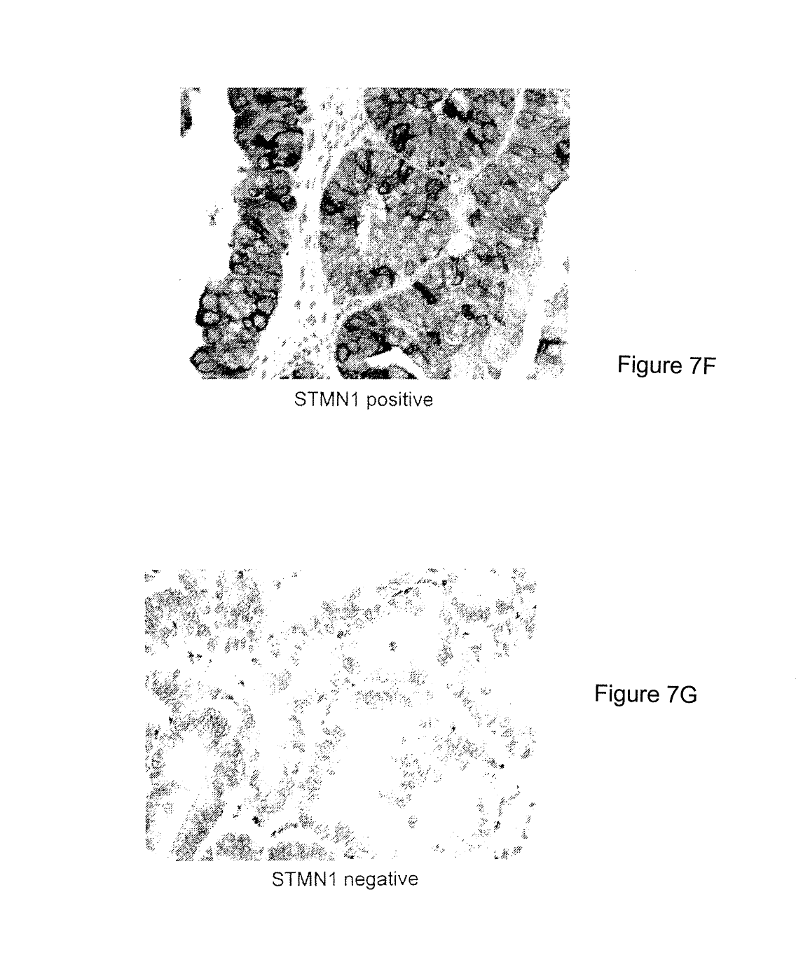

The finding that both PIK3CA amplification and the Cluster 2 expression profile indicate aggressive tumors, coupled with the association between PIK3CA amplification and the in vitro PI3K activation signature, suggested that the broader set of aggressive tumors in Cluster 2 might share the in vitro PI3K activation signature. This appears to be true: tumors in Cluster 2 without PIK3CA amplification have significantly higher PI3K scores than tumors in Cluster 1 (p<0.001) and equal to tumors with amplification of PIK3CA (FIG. 3F). Moreover, the Cluster 2 signature is highly anticorrelated with the signature of treatment with LY-294002 (p=0.02; FIG. 7A-B). Furthermore, tumors with high PI3K scores are associated with poor survival (p=validation datasets (p=0.01 and 0.001, respectively).

One possible cause of overexpression of the PI3K activation signature among tumors without PIK3CA amplification is decreased expression of the downstream PI3K pathway member PTEN. Decreased PTEN expression was associated with increased PI3K scores in both our test and validation datasets (p<0.001 and p=0.03 respectively), regardless of PIK3CA amplification status. Decreased PTEN expression was also associated with markers of aggressive disease (p=0.02).

Conversely, among the 45 tumors with expression data that we sequenced for PTEN, mutations did not associate with high PI3K scores (p=0.6; FIG. 7D). On the contrary, more mutations in the non-aggressive Cluster 1 than Cluster 2 (p=0.04; FIG. 1A) have been observed.

Overexpression and mutation of PIK3CA also appear to have different implications. Significantly higher PIK3CA expression in tumors with aggressive features, including those without PIK3CA amplification (p=0.05 and 0.0009 among test and validation data) have been found. However, among the 41 tumors with expression data that were sequenced for PIK3CA, mutations did not associate with high PI3K scores (p=0.8; FIG. 7E) or features of aggressive disease (p=0.5). Further, it cannot be confirm the finding that exon 20 mutations correspond to aggressive tumors (Catasus L et al. (2008) Mod Pathol 21:131-139) (data not shown). Although PIK3CA mutations have previously been noted primarily in endometrioid cancers (011ikainen M et al. (2007) Int J Cancer 121:915-920), no correlation with histologic subtype (p=1) has been found. These results were surprising in light of evidence that overexpression of mutated, but not wild-type, PIK3CA leads to transformation, and suggest either of 2 possibilities: 1) PIK3CA suffers from prevalent cryptic mutations, or 2) the effects of wild-type PIK3CA overexpression in human tumors were not captured by the transformation assays.

### Expression of the P13 Kinase Pathway Member STMN1 is an Independent Prognostic Indicator

The suggestion that PI3K activation associates with poor prognosis suggested that measuring PI3K activity might improve prognostication of localized endometrial cancer. Expression of STMN1 has previously been shown to correlate with PI3K activity in breast cancer (Saal LH et al. (2007) Proc Nati Acad Sci USA 104:7564-7569) and can be measured by immunohistochemistry in paraffin-embedded tissue. Herein, STMN1 expression by immunohistochemistry (FIG. 7F-G) in 72 tumors, including 66 with SNP array and 53 with expression data have been determined (FIG. 1A). Although STMN1 is not a member of our PI3K activation signature, STMN1 expression correlated with PI3K scores (p=0.05). High STMN1 expression also correlated with PIK3CA amplification (p=0.04) and overexpression (p=0.04), and segregation in Cluster 2 (p=0.03), supporting our prior associations between these features and PI3K pathway activation.

High STMN1 expression was also associated with poor recurrence-free survival in our original tumor set (p=0.006) and with poor recurrence-free (p=0.01) and overall (p=0.01) survival in a validation set of 241 tumors from a population-based series of all endometrial carcinoma in Hordaland County from 1981-1990 (Salvesen H B, Iversen O E & Akslen L A (1999) J Clin Oncol 17:1382-1390; Salvesen H B et al. (2002) Cancer 94:2185-2191). In both tumor sets, STMN1 expression correlated with grade, mitotic rate, presence of necrosis or vascular invasion, and Type II status (Table 5A). Nevertheless, across all 313 cases (minus 5 with missing clinical data), high STMN1 expression was an independent prognostic indicator to FIGO stage, histologic subtype, grade, and age (p=0.004; FIG. 3G, Table 5B). In particular, high STMN1 expression was associated with poor prognosis in the otherwise low-risk endometrioid subgroup (p=0.007, data not shown).

Ultimately, the goals of integrated genomic analyses of localized tumors are to enable development of clinical assays to distinguish aggressive tumors requiring therapy beyond resection, and of effective therapeutics for such tumors. It is shown herein that both transcriptional and copy-number profiles of endometrial tumors contain prognostic information that is partly reflected in expression levels of PIK3CA, in vitro PI3K activation signatures, PTEN, and STMN1. Further, it is shown that PTEN and PIK3CA mutations appear to have different transcriptional and phenotypic correlates than changes in expression of these genes. These results suggest that further investigation of the specific consequences of mutation and altered expression is warranted. They also emphasize the potential utility of clinical assays for PI3K pathway activation to identify patients with aggressive disease, and the particular relevance of therapeutics that inhibit this pathway.

Aside from the PI3K pathway, the general survey of chromosomal changes in endometrial carcinoma also identified approximately twenty other regions of significant copy-number change. Most of these copy-number changes involve tens to hundreds of genes, so even in cases where known oncogenes or tumor suppressors are within the regions most affected by these copy-number changes, the genomic data are ambiguous as to the actual target. In many cases, including amplification of 3q26.32, the size of these events may suggest multiple targets. Moreover, functional data tying even known oncogenes and tumor suppressors to carcinogenesis in endometrial cancer model systems are for the most part lacking. The limited number of significant regions of copy-number change suggests that comprehensive, systematic experiments to identify these oncogenes and tumor suppressors in endometrial cancer are feasible. Such experiments point to therapeutic targets for women with all stages of endometrial carcinoma.

Selecting Gene Subsets with Good Combined Discriminatory Power

As our objective function to minimize for determination of maximal predictive power, we used the sum of squared residuals between the relative probability of the correct class label and one given by a diagonal linear discriminant analysis (DLDA) classifier. The relative probability given a DLDA classifier is the probability density for the correct class divided by the sum of probability densities over both classes. Ideally a classifier assigns relative probability 1 to the correct class label in all cases, but this will often not be the case in practice.

We then tested increasing numbers of genes using a forward feature subset selection method (Jonassen, B. T., 2002, Genome Biology, 3:1-0017.11) and found a 29 gene predictor gave the best results. These genes were therefore included in a gene set for validation with QRT-PCR.

### Immunohistochemical Staining

5 um tissue microarray sections of paraffin-embedded tissue were stained, using antigen retrieval for 10 ruin at 750 W and 15 min at 350 W in Citrate buffer (pH=6). Slides were incubated 1 hour at room temperature with polyoclonal STMN1 antibody (#3352, Cell Signaling) diluted 1:50. A staining index was calculated as the product of staining intensity (0-3) and area of positive tumor cells (1=<10%, 2=10%-50%, 3=>50%). Values in the upper quartile (which corresponded to indices of 6 and 9) were considered positive.

The association between 3q26.32 amplification and tumor recurrence suggests a causal relationship, with its functional effects leading to the aggressive phenotype. An alternative model would be that both 3q26.23 amplification and the aggressive phenotype are caused by a prior event, such as generalized aneuploidy in the cell, leading to an association but no direct causal link. Although this possibility cannot be ruled out, when aneuploidy in 59 of the tumors with SNP array data have been analysed, it has been found that 3q26.32 amplification remains significantly associated with recurrence-free survival after adjustment for the impact of ploidy (p=0.03). It therefore appears that amplification of 3q26.32 has an association with poor survival independent of the overall level of copy-number changes in the cell.

Ploidy was determined from DNA histograms based on measurement of 10⁴-10⁵ cells by flow cytometry, using fresh tumors and adjacent HE sections to confirm malignant histology.

### Determining STMN1 Expression in Metastatic Endometrial Cancer

### Material and Methods

Between 2001 and September 2010, 603 patients treated for endometrial cancer were recruited prospectively in a population based setting, Stathmin expression in primary tumors were measured by immunohistochemistry and linked to treatment response to taxanes in patients with metastatic disease. Response was evaluated by the RECIST criteria and analysed as partial-/complete response versus stable disease/progression.

### Results

Of the 603 patients a total of 116 either relapsed (n=79) or progressed (n=37) after their first line of treatment. Of these, 90 were treated with chemotherapy (n=33), radiation (n=38) or hormonal therapy (n=15). The remaining did not receive any further treatment or underwent surgery. Complete information regarding response to therapy according to the RECIST criteria was available in 57 patients. Stathmin expression in primary tumors predicted response to microtubule-stabilising chemotherapy (p=0.02, FE test): Amongst patients with low expression of stathmin 11 of 12 (92%) had partial-/complete response, whereas only 2 of 6 (33%) patients with a high level of stathmin has partial-/complete response (p=−0.02, F.E.). Stathmin expression was not associated with response to other treatment modalities.

TABLE 1
Patient characteristics and histopathologic variables for the endo-
metrial carcinoma series studied compared with a population-based
patient series from the same region

| | General | Expression Arrays | | SNP Arrays | |
|---|---|---|---|---|---|
| Characteris- | Population | (n = 57) | | (n = 74) | |
| tics | (n = 285) | Raw value | P-value* | Raw value | P-value |
| Median age | 65 years | 63 years | 0.8 | 64 years | 0.8 |
| | (range, | (range, | | (range, | |
| | 33-92 | 39-91 | | 39-91 | |
| | years) | years) | | years) | |
| FIGO stage | 55 (19%) | 9 (16%) | 0.7 | 12 (16%) | 0.7 |
| III or IV | | | | | |
| Non- | 29 (10%) | 6 (11%) | 1 | 8 (11%) | 0.8 |
| endometrioid | | | | | |
| histology | | | | | |
| Histologic | 59 (21%) | 13 (23%) | 0.7 | 15 (21%) | 1 |
| grade 3 | | | | | |

″ Compared to general population, using a Fisher's exact test, except for age comparisons n which a Kruskal-Wallis test was used

TABLE 2
Differences in clinical and histopathologic characteristics between
clusters I and II and summarised gene mRNA predictor in microarray
and qPCR datasets

| Characteris- | Cluster 1 | Cluster 2 | | Microarray | qPCR |
|---|---|---|---|---|---|
| tic | (n = 29) | (n = 28) | P-value* | P-value* | P-value* |
| Median age | 62 years | 67 years | 0.6 | | |
| FIGO stage | 1 (3%) | 8 (29%) | 0.01 | 0.02 | 0.01 |
| III or IV | | | | | |
| Non- | 0 (0%) | 6 (21%) | 0.01 | 0.03 | 0.003 |
| endometrioid | | | | | |
| histology | | | | | |
| Histologic | 1 (3%) | 12 (43%) | <0.001 | 0.001 | 0.001 |
| grade 3 | | | | | |
| Depth of | 6¹″ | 8.5¹ | 0.06 | n.s. | 0.05 |
| myometrial | | | | | |
| infiltration | | | | | |
| Mitotic | | 10^{$} | 0.002 | 0.002 | 0.002 |
| figures | | | | | |
| Loss of | 4 (14%) | 12 (43%) | 0.02 | n.s. | 0.01 |
| estrogen | | | | | |
| receptor | | | | | |
| Loss of pro- | 2 (7%) | 11 (39%) | 0.005 | n.s. | 0.002 |
| gesterone | | | | | |
| receptor | | | | | |
| Presence of | 13 (45%) | 22 (76%) | 0.01 | 0.03 | 0.002 |
| necrosis | | | | | |
| Vascular | 6 (27%) | 16 (73%) | 0.007 | 0.02 | 0.002 |
| invasion | | | | | |
| Type II | 1 (3%) | 16 (57%) | <0.001 | <0.001 | <0.001 |
| tumor^{§} | | | | | |
| Aggressive | | | | <0.001 | <0.001 |
| cluster 2 | | | | | |

″Using a Fisher's exact test except for comparisons between continuous variables, in which a Mann-Whitney U test was used.
Median value in millimeters
Median number of mitotic figures per 10 fields at original magnification × 40
Defined as non-endometrioid, high-grade endometrioid, or lacking both estrogen receptor and progesterone receptor
n.s., not significant

TABLE 3
Univariate (UV) and multivariate survival analysis of recurrence-free
survival among 53 endometrial cancer patients cured by surgery and
adjusted hazard ratios (HRs) for recurrence among endometrial
carcinomas for cluster 2 and histopathologic variables

| | | | | Hazard Ratio | |
|---|---|---|---|---|---|
| Histopa- | Univariate | | | Histopa- | |
| thologic | Survival | Hazard Ratio | | thologic | |
| variable | P-value* | Cluster 2t | P-valuer | Variable | P-valuer |
| FIGO stage | n.s. | 3.0 (0.9.-10.4) | 0.08 | 1.3 (0.3-6.3) | 0.7 |
| III or IV | | | | | |
| Non- | 0.17 | 2.9 (0.8-10.2) | 0.1 | 1.6 (0.3-7.9) | 0.6 |
| endometrioid | | | | | |
| histology | | | | | |
| Histologic | 0.009 | 2.0 (0.5-8.2) | 0.3 | 2.9 (0.8-10.8) | 0.1 |
| grade 3 | | | | | |
| Loss of | n.s. | 3.2 (0.9-11.2) | 0.07 | 1.0 (0.3-3.3) | 0.9 |
| estrogen | | | | | |
| receptor | | | | | |
| Loss of pro- | 0.14 | 2.7 (0.7-10.2) | 0.1 | 1.5 (0.4-5.6) | 0.6 |
| gesterone | | | | | |
| receptor | | | | | |

*Log rank test.
tHazard ratio for cluster 2, adjusted for listed histopathologic variable in column 1; 95% confidence intervals in parentheses.
Using a log-ratio test.
⁵ Hazard ratio for histologic variable in column 1, adjusted for cluster segregation; 95% confidence intervals in parentheses.
n.s., not significant.

TABLE 4
List of peak regions of amplification, deletion, and non-overlapping
LOH

| | | | | | Oncogene |
|---|---|---|---|---|---|
| | | | | | or Tumor |
| | | | | | Suppressor |
| | | Boundaries | | Frequency^{t} | Gene in |
| | Cytoband | of Peak* | Q-value | (°/u) | Region |
| Amplification | | | | | |
| 1 | 1p34.3 | 30.1-41.9 | 2e−3 | 40 | |
| 2 | 1q42.13 | 224.8-237.7 | 3e−18 | 47 | LMYC1 |
| 3 | 3q26.32 | 173.3-184.9 | 1e−14 | 15 | PIK3CA |
| 4 | 6p21.2 | 37.5-38.3 | 0.11 | 15 | |
| 5 | 7p11.2 | 54.5-65.2 | 0.13 | 12 | EGFR |
| 6 | 8q24.21 | 122.6-129.7 | 4e−8 | 26 | MYC |
| 7 | 10q22.2 | 75.0-79.7 | 0.07 | 16 | |
| 8 | 12p12.1 | 25.5-27.8 | 0.02 | 12 | KRAS |
| 9 | 17q12 | 36.3-49.1 | 0.08 | 26 | ERBB2 |
| 10 | 19q12 | 34.0-35.6 | 4e−7 | 45 | CCNE1 |
| 11 | 20q13.2 | 45.3-57.7 | 2e−3 | 23 | AURKA |
| Deletion | | | | | |
| 1 | 71p36.32 | 0-19.3 | 0.24 | 10 | CHD5IPAX7 |
| 2 | 3p26.2 | 0-3.5 | 0.13 | 4 | FBXW7 |
| 3 | 4q34.3 | 92.1-183.3 | 8e−4 | 8 | |
| 4 | 6q16.3 | 10.48-10.49 | 0.04 | 9 | |
| 5 | 7p22.2 | 0-4.2 | 0.12 | 10 | |
| 6 | 7q33 | 109.2-137.1 | 0.11 | 4 | |
| 7 | 8p21.2 | 25.1-29.8 | 0.02 | 12 | |
| 8 | 11q23.3 | 116.5-134.5 | 0.18 | 8 | |
| 9 | 13q12.3 | 0-27.8 | 0.22 | 6 | |
| 10 | 15q26.3 | 97.9-100.3 | 0.07 | 10 | |
| 11 | 16q21 | 58.3-65.1 | 1e−3 | 18 | |
| 12 | 17p12 | 11.5-12.2 | 0.02 | 8 | |
| 13 | 22q13.2 | 41.7-44.3 | 0.18 | 10 | |

*Mb coordinates using hg16 build.
^{t}Frequency of amplification or deletion to any level. High-level amplifications were seen for LMYC, PIK3CA, EGFR, 6p21.2, EGFR, CCNE1 (1 case each), and MYC (2 cases).

TABLE 5
Correlations between amplifications and recurrence-free survival

| | | Number of | Mean | Mean | |
|---|---|---|---|---|---|
| | Onco- | Amplified | Surviva! | Surviva! | |
| | gene | Samples | Among | Among Un- | Bonferroni- |
| | in | (and Recur- | Amplified | ampflified | Corrected |
| Cytoband | Region | rences)* | Samples^{t} | Samples^{t} | P-Value^{s} |
| 1p34.3 | | 25 (5) | 4.2 | 4.7 | 1.0 |
| 1q42.13 | LMYC | 33 (8) | 4.2 | 4.9 | 0.9 |
| 3q26.32 | PIK3CA | 9 (4) | 2.4 | 4.8 | 0.03 |
| 6p21.2 | | 8 (3) | 2.6 | 4.7 | 0.4 |
| 7p11.2 | EGFR | 8 (2) | 3.7 | 4.6 | 1.0 |
| 8q24.21 | MYC | 16 (4) | 4.0 | 4.7 | 1.0 |
| 10q22.2 | | 10 (4) | 3.0 | 4.8 | 0.6 |
| 12p12.1 | KRAS | 7 (4) | 2.4 | 4.9 | 0.01 |
| 17q12 | ERBB2 | 16 (4) | 4.2 | 4.7 | 1.0 |
| 19q12 | CCNE1 | 29 (6) | 4.3 | 4.7 | 1.0 |
| 20q13.2 | AURKA | 14 (5) | 3.3 | 4.8 | 0.4 |

″ Number of samples amplified to any degree among the 68 competely resected tumors with follow-up clinical data, with number of recurrences among amplified samples (of 13 recurrences overall) in parentheses.
^{t}In years after primary surgery.
^{t}By log-rank test, after Bonferroni correction for 11 hypotheses.

TABLE 6
Ummunohistochemical Stathmin (STMN1) expression in a population-
based series of endometrial carcinomas: correlation with clinical phenotype

| Characteristic | Stathmin Negative | Stathmin Positive | P-value^{r} |
|---|---|---|---|
| Original tumor series* | n = 53 | n = 19 | |
| FIGO stage III or IV | 13% | 11% | n.s. |
| Non-endometrioid | 9% | 21% | 0.19 |
| histology | | | |
| Histologic grade 3 | 17% | 47% | 0.01 |
| Depth of myometrial | 6.5 | 7.5 | n.s. |
| infiltration* | | | |
| Mitotic rate* | 7 | 18 | 0.01 |
| Loss of ER | 25% | 32% | n.s. |
| Loss of PR | 19% | 53% | 0.005 |
| Presence of necrosis | 50% | 77% | 0.06 |
| Vascular invasion | 33% | 65% | 0.02 |
| Type II tumor | 26% | 58% | 0.01 |
| Aggressive cluster^{iT} | 44% | 75% | 0.03 |
| Validation series* | n = 175 | n = 66 | |
| FIGO stage III or IV | 17% | 21% | n.s. |
| Non-endometrioid | 9% | 15% | 0.18 |
| histology | | | |
| Histologic grade 3 | 14% | 32% | 0.001 |
| Depth of myometrial | 4 | 7 | 0.003 |
| infiltration* | | | |
| Mitotic rate* | 8 | 14 | <0.001 |
| Loss of estrogen | 18% | 45% | <0.001 |
| recaptor | | | |
| Loss of progesterone | 23% | 48% | <0.001 |
| receptor | | | |
| Presence of necrosis | 52% | 77% | <0.001 |
| Vascular invasion | 34% | 62% | <0.001 |
| Type II tumor^{s} | 35% | 64% | <0.001 |

*In the original tumor series, data are missing in 3 cases for histologic grade, depth of myometrial infiltration, mitotic rate, necrosis and vascular invasion, and for cluster annotation in 19 cases. In the validation series, data are missing in 1 case for FIGO stage, in 6 cases for estrogen receptor/progesterone receptor status, in 38 cases for depth of myometrial infiltration, and in 7 cases for type I/II classification
¹″ Using a Pearson chi-square test when otherwise not specified.
Median depth of myometrial infiltration in millimetres, number of mitotic figures per 10 fields at magnification 40 x, Mann-Whitney U test.
Defined as either non-endometrioid, high-grades endometrioid, or lacking both estrogen receptor and progesterone receptor.
^{IT}N = 53 cases, 1-sided test.
n.s., not significant.

TABLE 7
Immunohistochemical Stathmin (STMN1) expression in a population-
based series of endometrial carcinomas: multivariate survival analysis of
clinicopathologic variables

| Characteristic | Hazard Ratio* | P-value^{r} |
|---|---|---|
| Age* | 1.048 (1.024-1.072) | <0.001 |
| FIGO stage | | |
| I | 1 | <0.001 |
| II | 3.04 (1-36-6.77) | |
| III | 10.65 (5.87-19.31) | |
| IV | 48.74 (20.74-114.57) | |
| Histologic subtype | | |
| Endometrioid | 1 | 0.2 |
| Non-endometrioid | 1.59 (0.79-3.23) | |
| Histologic grade | | |
| 1 | 1 | 0.38 |
| 2 | 1.73 (0.75-3.96) | |
| 3 | 1.36 (0.50-3.76) | |
| STMN1 staining | | |
| Negative | 1 | 0.004 |
| Positive | 2.14 (1.28-3.59) | |

″ Hazard ratio with 95% confidence intervals in parentheses, based on the Cox proportional hazards model.
^{t}Using a log-ratio test.
*Continuous variable with hazard ratio given per year.

1. A method for the diagnosis or differentiation of endometrial carcinoma in an individual comprising the step of determining alterations, in particular, amplifications and deletions, of chromosomal regions 3q26.32 and 12p12.1, alterations of the gene expression profile of the genes (gene signature): upregulation of the genes PLEKHK1, ATP10B, NMU, MMP1, ATAD2, NETO2, TNNI3, PHLDA2, OVOL1 and down-regulation of the genes: NDP, KIAA1434, MME, CFH, MOXD1, SLC47A1, RBP1, PDE8B, ASRGL1, ADAMTS19, EFHD1, ABCA5, NPAS3, SCML1, TNXB, ENTPD3, AMY1A, ENPP, RASL11B, PDZK3, or the expression status of the STMN1 gene or protein.

2. A method for evaluating the probability of survival for an individual suffering from endometrial carcinoma or the clinical outcome comprising the step of determining alterations, in particular, amplifications and deletions, of chromosomal regions 3q26.32 and 12p12.1, alterations of the gene expression profile of the genes (gene signature): upregulation of the genes PLEKHK1, ATP 10B, NMU, MMP1, ATAD2, NETO2, TNNI3, PHLDA2, OVOL1 and down-regulation of the genes: NDP, KIAA1434, MME, CFH, MOXD1, SLC47A1, RBP1, PDE8B, ASRGL1, ADAMTS19, EFHD1, ABCA5, NPAS3, SCML1, TNXB, ENTPD3, AMY1A, ENPP, RASL11B, PDZK3, or the expression status of the STMN1 gene or protein.

3. The method for diagnosing or differentiation of endometrial carcinoma in an individual according to claim 1 comprising the steps of determining the PI3K activity in patients having aggressive endometrial carcinoma.

4. The method according to claim 3 wherein an elevated P13K activity is associated with the high grade aggressive phenotype of endometrial cancer.

5. Method for the diagnosis or differentiation of endometrial carcinoma in an individual according to claim 1, comprising the step of determining the expression status of STMN1 in said individual for allowing diagnosis and differentiation of endometrial carcinoma.

6. The method according to claim 5 whereby high STMN1 expression is associated with poor recurrence free survival and overall survival in patients suffering from endometrial carcinoma.

7. A method according to claim 1 allowing differentiation between high grade aggressive phenotype and low grade phenotype of endometrial carcinoma whereby high STMN1 expression is associated with high grade aggressive phenotype of endometrial carcinoma.

8. The method according to claim 1 comprising the step of determining expression of the STMN1 gene in combination with determining at least one of the alterations in the chromosomal regions 3q26.32 and 12p12.1, or in combination with determining alterations of the gene expression profile of the genes (gene signature): upregulation of the genes PLEKHK1, ATP10B, NMU, MMP1, ATAD2, NETO2, TNNI3, PHLDA2, OVOL1 and down-regulation of the genes: NDP, KIAA1434, MME, CFH, MOXD1, SLC47A1, RBP1, PDE8B, ASRGL1, ADAMTS19, EFHD1, ABCA5, NPAS3, SCML1, TNXB, ENTPD3, AMY1A, ENPP, RASL11B, PDZK3.

9. A method for diagnosing or identifying endometrial carcinoma with high grade aggressive phenotype in a subject comprising
a) determining the level or amount of STMN1 in a sample of said subject; and
b) comparing the level or amount determined in step a) to a reference value, wherein an increase in the level or amount relative to the reference value is indicative for endometrial carcinoma with high grade aggressive phenotype.

10. A method for the stratification of the therapy of endometrial tumor, ovarian cancer, breast cancer, non-small lung cancer or hormone refractory prostate cancer in an individual or monitoring therapeutic efficacy of said diseases in an individual comprising the step of determining the expression status of the STMN1 gene or protein and stratifying the therapy or monitoring the efficacy of therapy of the endometrial tumor, ovarian cancer, breast cancer, non-small lung cancer or hormone refractory prostate cancer in said individual.

11. A method for the stratification of the therapeutic regimen or monitoring the therapeutic efficacy of an individual with endometrial carcinoma, ovarian cancer, breast cancer, non-small lung cancer or hormone refractory prostate cancer according to claim 10 comprising
a) determining the level or amount of STMN1 in a sample of said individual and
b) determining the therapeutic regimen or monitoring the therapeutic efficacy based on the level or amount of STMN1.

12. A method for monitoring the progression of endometrial carcinoma, ovarian cancer, breast cancer, non-small lung cancer or hormone refractory prostate cancer in an individual comprising
a) detecting the level or amount of STMN1 in a first sample from the individual at a first time point;
b) determining the level or amount of STMN1 in a second sample from the individual at a second point in time; and
c) comparing the level or amount of STMN1 determined in step a) to the level or amount detected in step b) or to a reference value.

13. The method according to claim 1 wherein determining the expression status of the gene STMN1 is effected on nucleic acid or amino acid level in said individual.

14. A kit for use in providing a diagnosis or differentiation of endometrial carcinoma in an individual, for the stratification of endometrial tumor therapy in an individual, monitoring therapeutic efficacy in an individual, or for evaluating the probability of survival for an individual suffering from endometrial carcinoma comprising means for determining determining alterations, in particular, amplifications and deletions, of chromosomal regions 3q26.32 and 12p12.1, alterations of the gene expression profile of the genes (gene signature): upregulation of the genes PLEKHK1, ATP10B, NMU, MMP1, ATAD2, NETO2, TNNI3, PHLDA2, OVOL1 and down-regulation of the genes: NDP, KIAA1434, MME, CFH, MOXD1, SLC47A1, RBP1, PDE8B, ASRGL1, ADAMTS19, EFHD1, ABCA5, NPAS3, SCML1, TNXB, ENTPD3, AMY1A, ENPP, RASLI1B, PDZK3, or the expression status of the STMN1 gene or protein.

15. A kit according to claim 14 wherein said means are means for determining the P13K activity in patients having aggressive endometrial carcinoma.

16. A kit for use in providing diagnosis or differentiation of endometrial carcinoma in an individual, for the stratification of therapy in an individual, or monitoring therapeutic efficacy in an individual according to claim 14 comprising means for detecting STMN1 expression status.

17. The method according to claim 9 for predicting the response to taxanes in an individual.

18. The method according to claim 9 for stratification of therapy for the treatment of metastatic endometrial cancer.

19. The method according to claim 10 wherein the endometrial tumor therapy is a therapy based on P13K inhibitors, Akt inhibitors, or mTOR inhibitors or PTEN activators.

20. A kit for use in a method according to claim 9.
